(19) United States
(12) Patent Application Publication
(10) Pub. No.: US 2005/0119461 A1
(43) Pub. Date: Jun. 2, 2005

(54) Novel receptor, and compounds which bind thereto

(75) Inventors: William J. Louis, Victoria (AU); Graham P. Jackman, Victoria (AU); Elizabeth L. Conway, Victoria (AU); Andrew L. Gundlach, Victoria (AU); Dimitri Iakovidis, Victoria (AU); Paul R. King, Victoria (AU); Simon N.S. Louis, Victoria (AU); Tracy Nero, Victoria (AU)

Correspondence Address: SUGHRUE MION, PLLC, 2100 PENNSYLVANIA AVENUE, N.W., SUITE 800, WASHINGTON, DC, 20037, US

(73) Assignee: THE UNIVERSITY OF MELBOURNE

(21) Appl. No.: 10/981,647

(22) Filed: Nov. 5, 2004

(30) Foreign Application Priority Data
Nov. 5, 1997 (AU) PP 0202

(51) Int. Cl. C07D 263/02; C07H 21/04; C07K 14/705; A61K 31/421

(52) U.S. Cl. 530/350; 536/23.5; 548/215; 514/374

(57) ABSTRACT

The present invention relates to a novel receptor, in particular to a new type of receptor with affinity for compounds of the oxazoline class, compounds which bind to this receptor, and the use of these compounds in the treatment of diseases, especially diseases of the central nervous system, the cardiovascular system and the kidney.

The present invention relates to a novel receptor, in particular to a new type of receptor with an affinity for compounds of the oxazoline class, compounds which bind to this receptor, and the use of these compounds in the treatment of diseases, especially diseases of the central nervous system, the cardiovascular system and the kidney.

Some compounds acting at this receptor were previously thought to be acting at a receptor which was a member of the family of receptors known as imidazoline (I)-receptors. It has now been found that these compounds also act at this novel receptor which is a separate distinct and specific site distinguishable from the known α₂-adrenoceptors and imidazoline I₁- and I₂-receptors.

Many imidazoline derivatives and structurally-related compounds including oxazolines and guanidines bind not only to α₂-adrenoceptors but also to I-receptors which differ in structure, function and distribution to α₂-adrenoceptors (Parini et al, 1996, Regunathan and Reis, 1996). Based on pharmacological studies, the I-receptor binding sites (I-RBS) have been classified into I₁- and I₂-receptor subtypes: an I₁-subtype with a high affinity for [³H]clonidine and an I₂-subtype with a low affinity for clonidine and a high affinity for the imidazoline radioligand, [³H]idazoxan (Parini et al, 1996; Regunathan and Reis, 1996).

I₂-RBS have been usually labelled in tissue membranes or sections using [³H]idazoxan, after masking for α₂-adrenoceptors, however, recent studies have shown that the new, high affinity radioligand [³H]2-BFI labels I₂-RBS in brain and kidney, and has a greatly enhanced selectivity for I₂-RBS over α₂-adrenoceptors compared to [³H]idazoxan (Hosseini et al, 1997; Lione et al, 1996). In rat brain the highest densities of I₂-RBS are found in the ependymal layer surrounding the ventricles, the arcuate and interpeduncular nuclei and specific nuclei of the brainstem (Mallard et al, 1995; Tesson et al, 1995). However, moderate levels of binding are also present throughout the cortex, basal ganglia, septum, hippocampus and amygdala (Mallard et al, 1995; Tesson et al, 1995). I₂-RBS have been localised to the outer mitochondrial membrane and in brain appear to be associated with astrocytes specifically through an association with the mitochondrial enzyme, monoamine oxidase B (MAO-B) (Tesson et al, 1995; Regunathan et al, 1993). A primarily glial localisation of I₂-receptors is further supported by findings that I₂-RBS are present on cultured cortical astrocytes but not neurons (Sastre and Garcia-Sevilla, 1993) and that in human brain I₂-RBS increase with age, compared to a decline in α_{2A}-adrenoceptors, consistent with known glial hypertrophy and loss of cortical noradrenergic projections (Olmos et al, 1994).

Rilmenidine, an oxazoline, is an antihypertensive drug similar to clonidine but without its pronounced sedative effects. It has been reported as binding to both I₁- and I₂-like receptors as well as α₂-adrenoceptors (Parini et al, 1996; Evans, 1996). In some reports its non adrenergic binding has been shown to be atypical to other I compounds (King et al, 1995). However compounds of high specificity for this binding site have now been unexpectedly identified, and this together with a demonstration of specific upregulation of these sites confirm it as neither an I₁- or I₂-receptor site, but as a distinct Oxazoline receptor. It has now been further established that specific I₁ and I₂-receptor compounds have poor or no detectable activity at the Oxazoline receptor confirming the distinct identity of this site.

The specific physiological upregulation of this new receptor has been demonstrated in the rat hippocampus and its presence has been confirmed in human brain. This receptor prefers compounds of the oxazoline class and it will be herein referred to as an oxazoline or Ox receptor.

Accordingly in a first aspect of the present invention there is provided a compound of the formula I: 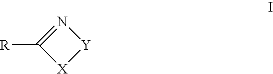
where R is the residue of an organic compound, X is O or S and Y is a divalent group making up a 5 or 6 membered ring which compound has a selectivity for the Ox receptor over one or both of the α₂- and I₂-receptors of greater than 1. Preferably R is connected to the heterocyclic ring via a divalent bridging group.

Preferably the compound of formula I has a selectivity of greater than 2, more preferably greater than 3, and most preferably greater than 5 over one or more of the α₂- and I₂-receptors.

Preferably the compound of formula I has a selectivity of greater than 1, more preferably greater than 2, over both of the α₂- and I₂-receptors.

It is also preferable that the compound of formula I has a selectivity for the Ox receptor area the I receptor of greater than 1, more preferably greater than 2 and most preferably greater than 3.

The term “selectivity” as used herein refers to the selectivity of a compound for the Ox receptor over another receptor in mammalian tissue containing said receptors. The selectivity is the reciprocal of the ratio of the affinities (in nM) for each receptor site. Preferably the tissue is human tissue.

Preferably the binding affinities for the receptors are as determined using the following ligands.
Ox: ³H-rilmenidine (preferably in the presence of excess adrenaline to block α-sites).
α₂: ³H-methoxyidazoxan
I₂: H-idazoxan (preferably in the presence of excess adrenaline to block α-sites).
I₁: ³H-clonidine (preferably in the presence of excess adrenaline to block α-sites).

Other suitably labelled ligands may also be used.

The Ox receptor is widely distributed in brain, particularly in the pineal gland, cortex, hippocampus, brain stem and spinal cord. In the hippocampus it is found in areas associated with memory, in particular the CA1-CA2 neurons i.e the pyramidal cells. In brain stem and spinal cord, the receptor is found in motor neurons and in brain stem nuclei such as the area postrema, rostroventrolateral medulla, nucleus tractus solitarius and locus coeruleus. These nuclei are associated with regulation of blood pressure. In kidney they are widely distributed in renal cortex and the outer stripe of the outer medulla, sites which are involved in regulation of ion transport. In the adrenal gland they are found in the cortex and the medulla.

Accordingly in a further aspect of the present invention there is provided a method for the treatment or prevention of diseases of the central nervous system, cardiovascular system or the kidney, or diseases associated with abnormal adrenal gland secretion which comprises administering an effective amount of a compound of formula I or a pharmaceutically acceptable salt or ester thereof to a subject in need thereof.

Diseases of the central nervous system include, but are not limited to, dementia, mood disturbances, degenerative conditions, such as stroke or aging, ischaemia, CNS trauma and neurodegenerative diseases, such as Alzheimer's disease and Parkinson's disease.

Diseases of the cardiovascular system include, but are not limited to, hypertension and ischaemic heart disease.

Diseases of the kidney include, but are not limited to, those diseases which affect renal tubular function.

Diseases associated with abnormal adrenal gland secretions include, but are not limited to, hypertension, heart failure and oedema.

The compounds of formula I may also be useful in the treatment of other diseases or conditions including, but not limited to, hyperglycaemia, glaucoma and peptic ulcer, and may be useful in producing effects such as analgesia.

Accordingly in a further aspect of the present invention there is provided a pharmaceutical composition for the treatment or prevention of diseases of the central nervous system, the cardiovascular system or the kidney, or diseases associated with abnormal adrenal gland secretion which comprises a compound of formula (I) or a pharmaceutically acceptable ester or salt thereof together with a pharmaceutically acceptable carrier or diluent.

The invention also provides the use of a compound of formula I in the manufacture of a medicament for the treatment or prevention of diseases of the central nervous system, the cardiovascular system or the kidney, or diseases associated with abnormal adrenal gland secretion.

In yet another aspect of the present invention there is provided the use of a compound of formula I as an agonist or antagonist to the Ox receptor.

In a preferred embodiment the compound of formula (I) is a compound of formula II: 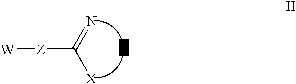
W is optionally substituted aryl, optionally substituted C₃-C₇ cycloalkyl or —CHR¹R² where R¹ and R² are independently selected from hydrogen, optionally substituted C₁-C₆ alkyl, optionally substituted C₃-C₇ cycloalkyl, optionally substituted aryl, and OR′ where R′ is optionally substituted aryl, optionally substituted C₃-C₇ cycloalkyl or optionally substituted C₁-C₆ alkyl, provided that both of R¹ and R² are not both hydrogen.
Z is —NH—, C₁-C₂ alkylene, —CH₂NH— or —CH₂CH₂NH—,
X is O or S, and
Y is optionally substituted C₂-C₄ alkylene, provided W is not OR′ when Z is NH or NHCH₂.

Preferably W is aryl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy); C₁-C₆ cycloalkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C_{1-C}₆ alkynyl or aryloxy); or —CHR¹R² where R¹ and R² are independently selected from hydrogen, C₁-C₆ alkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy), C₃-C₆ cycloalkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy), aryl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy), and OR where R is aryl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy), C₃-C₆ cycloalkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy), or C₁-C₆ alkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy); provided R¹ and R² are not both hydrogen.

More preferably W is phenyl or cyclohexyl, naphthyl, each of which may be optionally substituted with one to three substituents selected from hydroxy, methoxy, ethoxy, benzyloxy, NO₂, NH₂, halogen, methyl and ethyl; or —CHR¹R² where R¹ and R² are independently selected from phenyl, naphthyl, cyclohexyl, cyclopentyl, cyclobutyl, cyclopropyl, methyl, ethyl, propyl and butyl, each of which may be optionally substituted with hydroxy, methoxy, ethoxy, benzyloxy, NO₂, NH₂, halogen, methyl and ethyl; provided R¹ and R² are not both hydrogen.

Preferably Z is imino.

Preferably X is oxygen.

Preferably Y is C₂-C₄ alkylene optionally substituted with C₁-C₄ alkyl, C₃-C₆ cycloalkyl, C₁-C₆ alkanoyloxy or C₁-C₆ alkyloxycarbonyl, or with two substituents which join together to form a 5-6 membered carbocyclic or heterocyclic ring. The carbocyclic or heterocyclic ring may be an optionally substituted, non-aromatic, aromatic or psuedoaromatic ring having 2 to 20 ring atoms.

More preferably, Y is unsubstituted C₂-C₄ alkylene, most preferably unsubstituted ethylene.

In another preferred embodiment the compound of formula (I) is a compound of formula III 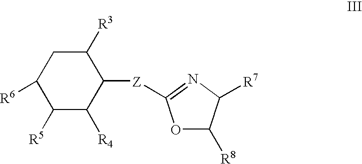
wherein R³, R⁴, R⁵ and R⁶ are independently selected from hydrogen, hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl and aryloxy.
Z is imino, C₁-C₂ alkylene, or —CH₂CH₂NH—.
R⁷ and R⁸ are independently selected from hydrogen, C₁-C₄ alkyl, C₃-C₆ cycloalkyl, C₁-C₆ alkanoyloxy or C₁-C₆ alkyloxycarbonyl or R⁷ and R⁸ may together form a 5 or 6 membered aromatic or non-aromatic carbocyclic or heterocyclic ring.

In yet another preferred embodiment the compound of formula (I) is a compound of formula IV: 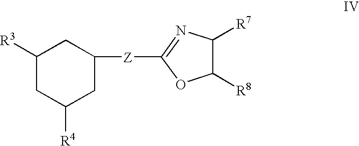
where R³, R⁴, R⁷, R⁸ and Z are as defined above in relation to formula III.

In yet another preferred embodiment the compound of formula (I) is a compound of formula V: 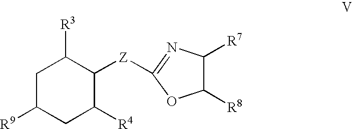
where R³, R⁴, R⁷, R⁸ and Z are as defined above in relation to formula III and R⁹ is C₁-C₄ alkyl or C₁-C₄ alkoxy.

In yet another embodiment the compound of formula (I) is a compound of formula VI: 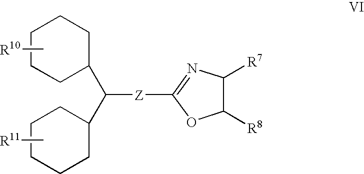
where R⁷, R⁸ and Z are as defined above in relation to formula III and R¹⁰ and R¹¹ are independently selected from hydrogen, hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl, aryloxy.

In a further embodiment of the compound of formula (I) is a compound of formula VII: 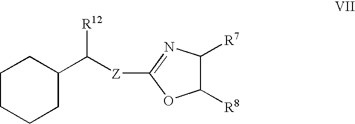
where R⁷, R⁸ and Z are as defined in relation to formula III and R¹² is hydrogen or optionally substituted C₁-C₆ alkyl, optionally substituted C₃-C₇ cycloalkyl or optionally substituted aryl.

In another embodiment the compound of formula (I) is a compound of formula VIII: 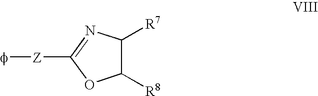
where φ is optionally substituted aryl and R⁷, R⁸ and Z are as defined above in relation to formula III.

Preferably φ is phenyl or naphthyl either of which may have one to four substituents selected from hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl. C₂-C₆ alkynyl and aryloxy.

In yet another embodiment the compound of formula (I) is a compound of formula IX: 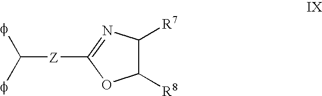
where R⁷, R⁸, Z and φ are as defined in relation to formula VIII.

In another embodiment the compound of formula I is a compound of formula X: 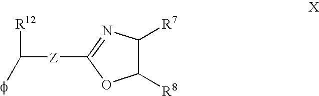
where R⁷, R⁸, R¹² and Z are as defined in relation to formula VII and φ is defined in formula VIII.

Preferably in formula III to X, Z is —NH— and R⁷ and R⁸ are hydrogen.

The term “aryl” as used herein refers to aromatic rings or ring systems containing between 3 and 20 carbon atoms. The rings or ring systems may be optionally substituted.

The term “aromatic rings or ring system” as used herein refers to a compound or substituent which includes or consists of one or more aromatic or pseudoaromatic rings. The rings may be carbocyclic, or heterocyclic, and may be mono or polycyclic ring systems. Examples of suitable rings include but are not limited to benzene, biphenyl, terphenyl, quaterphenyl, naphthalene, tetrahydronaphthalene, 1-benzylnaphthalene, anthracene, dihydroanthracene, benzanthracene, dibenzanthracene, phenanthracene, perylene, pyridine, 4-phenylpyridine, 3-phenylpyridine, thiophene, benzothiophene, naphthothiophene, thianthrene, furan, pyrene, isobenzofuran, chromene, xanthene, phenoxathin, pyrrole, imidazole, pyrazole, pyrazine, pyrimidine, pyridazine, indazole, indole, indolizine, isoindole, purine, quinoline, isoquinoline, phthalazine, quinoxaline, quinazoline, pteridine, carbazole, carboline, phenanthridine, acridine, phenanthroline, phenazine, isothiazole, isooxazole, phenoxazine and the like, each of which may be optionally substituted. The term “aromatic rings or ring system” includes molecules, and macromolecules, such as polymers, copolymers and dendrimers which include or consist of one or more aromatic rings. The term “pseudoaromatic” refers to a ring system which is not strictly aromatic, but which is stabilized by means of delocalization of π electrons and behaves in a similar manner to aromatic rings. Examples of pseudoaromatic rings include but are not limited to furan, thiophene, pyrrole and the like.

In this specification “optionally substituted” means that a group may or may not be substituted with one or more non deleterious groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, isocyano, cyano, formyl, carboxyl, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, imino, alkylimine, alkenylimine, alkynylimino, arylimino, benzylimino, dibenzylanino, acyl, alkenylacyl, alkynylacyl, arylacyl, acylamino, diacylamino, acyloxy, alkylsulphonyloxy, arylsulphenyloxy, heterocyclyl, heterocycloxy, heterocycliamino, haloheterocyclyl, alkylsulphenyl, arylsulphenyl, carboalkoxy, carboaryloxy mercapto, alkylthio, benzylthio, acylthio, sulphonamido, sulfanyl, sulfo and phosphorus-containing groups.

The term “alkyl” as used herein alone or in compound words such as “alkylester” denotes straight or branched chain alkyl, preferably C₁-C₂₀ alkyl. Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2,-trimethylpropyl, 1,1,2-trimethylpropyl, heptyl, 5-methoxyhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3,-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyl-octyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propylocytl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like.

The term “cycloalkyl” as used herein denotes cyclic alkyl groups, including mono- and polycyclic alkyl groups. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Preferably the cyclic alkyl groups are C₃-C₆ alkyl groups such as cyclopentyl and cyclohexyl. Examples of cyclic alkyl also including heterocyclic forms containing one of oxygen, sulphur and nitrogen atoms such as tetrahydropyran, tetrahydrofuran, pyrimidines, pyridizines, oxazines and related compounds.

The term “alkoxy” denotes straight chain or branched alkoxy, preferably C₁₋₂₀ alkoxy. Examples of alkoxy include methoxy, ethoxy, n-propoxy, isopropoxy and the different butoxy isomers.

The term “alkenyl” denotes groups formed from straight chain, branched or cyclic alkenes including ethylenically mono-, di- or poly-unsaturated alkyl or cycloalkyl groups as previously defined, preferably C₂₋₂₀ alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl.

The term “alkynyl” denotes groups formed from straight chain, branched or cyclic alkyne including alkyl and cycloalkyl groups as previously defined which contain a triple bond, preferably C₂₋₂₀ alkynyl. Examples of alkynyl include ethynyl, 2,3-propynyl and 2,3- or 3,4-butynyl.

The term “acyl” either alone or in compound words such as “acyloxy”, “acylthio”, “acylamino” or “diacylamino” denotes carbamoyl, aliphatic acyl group and acyl group containing an aromatic ring, which is referred to as aromatic acyl or a heterocyclic ring which is referred to as heterocyclic acyl, preferably C₁₋₂₀ acyl. Examples of acyl include carbamoyl; straight chain or branched alkanoyl such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; alkoxycarbonyl such as methoxycarbonyl, ethoxycarbonyl, t-butoxycarbonyl, t-pentyloxycarbonyl and heptyloxycarbonyl; cycloalkylcarbonyl such as cyclopropylcarbonyl, cyclobutylcarbonyl, cyclopentylcarbonyl and cyclohexylcarbonyl; alkylsulfonyl such as methylsulfonyl and ethylsulfonyl; alkoxysulfonyl such as methoxysulfonyl and ethoxysulfonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aralkoxycarbonyl such as phenylalkoxycarbonyl (e.g. benzyloxycarbonyl); aryloxycarbonyl such as phenoxycarbonyl and napthyloxycarbonyl; aryloxyalkanoyl such as phenoxyacetyl and phenoxypropionyl; arylcarbamoyl such as phenylcarbamoyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolylglyoxyloyl and thienylglyoxyloyl.

The term “heterocyclic” as used herein refers to aromatic, pseudo-aromatic and non-aromatic rings or ring systems which contain one or more heteroatoms selected from N, S and O and which may be optionally substituted. Preferably the rings or ring systems have 3 to 20 carbon atoms.

Where a carbon is asymmetrically substituted and optical isomers exist for compounds of formula I it has been found that the optical isomers vary in their affinity and selectivity for the Ox receptor and α₂-adrenoceptor's as much as 100-fold.

Free base forms of the compounds of the invention may be converted into the acid addition salts in a conventional manner and vice versa. Suitable acids for addition salts include, hydrochloric, tartaric, malonic and fumaric.

In the compounds of the invention the carbon atom next to Z may be asymmetrically substituted as may the carbons of the oxazoline ring. The compounds may be prepared as racemic mixtures or the isomers may be isolated in the conventional manner from asymmetric starting materials or by resolution of the base on fractional crystallisation with optically active acids.

A compound of formula I, where X is O, Y is CH₂CH₂, and R is connected to the heterocyclic ring via a bridging group —NH—, may be prepared in two steps by converting the appropriate primary amine into an oxazoline via the chloroethyl urea.

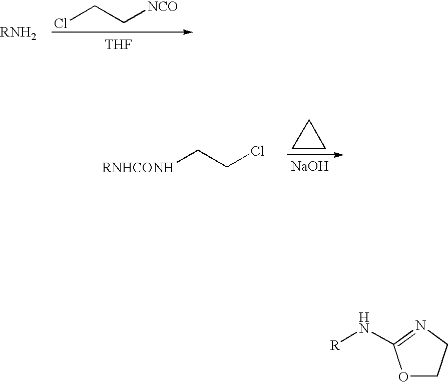

Other compounds may be prepared in analogous manner.

Some of the compounds of formula I are novel and represent a further aspect of the present invention.

Some compounds of formula II and III are known and are excluded from this further aspect of the invention.

Compounds with the general formulae III and V where Z is —NH—, R⁵, R⁶, R⁷ and R⁸ are H, R³ is lower alkyl of 1 to 3 carbon atoms and R⁴ and R⁹ are independently H or a lower alkyl of 1 to 5 carbon atoms have been previously disclosed in US3598833 and US5034406 to be useful in the following therapeutic areas: reducing or maintaining intra-ocular pressure, for example in the treatment of glaucoma; increasing blood-sugar levels, for example in the treatment of hyperglycemia; inhibiting gastric-secretion, for example in the treatment of ulcers and hypertonia; vasoconstriction; depressing blood pressure; sedation; local anesthesia and hypertension.

Compounds with the general formula II where Z is —NH—, X is O, Y is —CH₂CH₂— and W is CHR¹R² where R¹ and R² can each independently be H (but not both H), or a low molecular weight alkyl group substituted with an optionally substituted cyclopropyl have been previously disclosed in U.S. Pat. No. 3,988,464 and U.S. Pat. No. 4,102,890 to be useful in the following therapeutic areas: in treating hypertension; treating heart failure and edematous disease; analgesia; depressing the cardiovascular system; neuromodulation; lowering blood pressure; treating cardiovascular diseases; treating neuropsychological disturbances; depression of the CNS which may result in hypnosis, analgesic action and or antipsychotic effects.

Compounds with the general formula II where Z is —NH—, X is O, Y is —CH₂CH₂— and W is CHR¹R² where R¹ can be a haloalkyl of 1 to 3 carbon atoms and R² can be a lower alkyl radical having 1 to 6 carbon atoms in a straight or branched chain, a lower cycloalkyl radical having from 3 to 7 carbon atoms which may be substituted by one or more lower alkyl radicals, or lower alkoxy radicals have been previously disclosed in U.S. Pat. No. 4,378,366 and DE4325491 to be useful in the following therapeutic areas: in treating hypertension; treating acute alterations in intermediary metabolism subsequent to surgery, trauma and burns.

Compounds with the general formula II where Z is —NH—, X is O, Y is —CH₂CH₂— and W is CHR¹R² where R₁ can be a haloalkyl of 1 to 3 carbon atoms and R₂ can be an optionally substituted phenyl or furanyl or thiophene or pyrrole group have been previously disclosed in U.S. Pat. No. 4,267,345 to be useful as antihypertensive agents.

The compound with the general formula II where Z is —NH—, X is O, Y is —CH₂CH₂— and W is CHR¹R² where R¹ and R² are cyclopropyl groups has been previously disclosed in patents U.S. Pat. No. 5,605,911, U.S. Pat. No. 5,459,133 and DE4325491 to be useful in the following therapeutic areas: treating post aggression syndrome, preventing neurotoxic side-effects when administered with an NMDA antagonist; combatting a naturally occurring form of NMDA-receptor hypofunction which occurs as a causative or aggravating mechanism in people suffering from schizophrenia; reduction of at least one neurotoxic side effect selected from the group consisting of formation of vacuoles in neurons in cerebrocortical or limbic brain regions, expression of heat shock proteins in cerebrocortical or limbic brain regions, alteration or loss of mitochondria in neurons, neuronal death or hallucinations and psychotomimetic effects; increasing the accommodative ability of the eye for the purpose of decreasing the severity of presbyopsia.

Homogenate binding studies have shown that I₂-RBS increase with age in human brain, consistent with glial hypertrophy. In autoradiographic studies we have compared the spatial relationship of I₂-RBS and Ox receptor sites and astrocytes in young and aged rats. At a level through the rostral hippocampus [³H]2-BFI (a specific I₂-receptor ligand) binding was not altered in the brain of 24 month-old rats compared to 3 month-old rats, although GFAP mRNA levels were elevated throughout both white and grey matter areas. Unexpectedly there was a dramatic difference in the pattern of Ox receptors. Binding to this receptor in the pyramidal cell layer of the CA1-CA3 region of the hippocampus was more prominent in the aged rats and there was a marked concentration of binding over the CA2 region. Additional binding was evident in layer 3 of the parietal cortex, the ventromedial and dorsomedial hypothalamic nuclei and the medial amygdaloid nuclei in the aged animals. This binding pattern distinct from that of GFAP mRNA suggests the binding is localised to neurons rather than to glial cells and provides further evidence of a distinct neuronal receptor site that is physiologically upregulated.

Short periods of global cerebral ischaemia induce selective neuronal loss in the CA1 field of the hippocampus in experimental models in animals and in humans after cardiac arrest (Petito et al, 1990). This delayed neuronal death has been widely studied in the rat four-vessel occlusion (4-VO) model and is accompanied by a reactive astrocytosis involving cell hypertrophy and hyperplasia and transformation of GFAP-negative into GFAP-positive glia that persist for some weeks post-ischaemia (Gustafson et al, 1990). It was originally hypothesised that I₂-RBS might be linked to this altered expression of GFAP, however, it was found when examining the spatial relationship of changes in I₂-RBS and reactive gliosis following 4-VO ischaemic injury in rats the pattern of binding of the selective I₂-RBS ligand, [³H]2-BFI, was unaltered. Unexpectedly, specific binding to Ox receptors was increased by approximately 60%, 4 and 8 days after the ischaemic insult but had returned to normal levels at later time points. This increase was observed only in the CA1 pyramidal cell region of the hippocampus and was not related to reactive astrocytosis and was localized to neuronal cell bodies. The upregulation of Ox receptors following ischaemia demonstrates for the first time that these Ox sites are involved in pathophysiological processes in the brain which appear to represent compensatory mechanisms in surviving neurons and provide a rationale for use of compounds which act at these sites as neuroprotective agents. The changes in Ox receptors also provide further evidence that the Ox receptor is a novel receptor site, distinct from I₂-receptors and a₂-adrenoceptors.

The Ox receptor pharmacophore as depicted by formula I differs from those of the I₂-receptor (Nero et al, 1997) and α₂-adrenoceptor (Timmermans and van Zwieten, 1977) in the nature of the bridging moiety Z and the hydrogen bond donor (or acceptor) groups (—NH— and X). For both the Ox receptor and a₂-adrenoceptor pharmacophores the bridging moiety Z induces a bend into the molecular configuration, whereas in the I₂-receptor pharmacophore Z, typically a bond, maintains an overall linear molecular configuration. The nature of the hydrogen bond donor (or acceptor) groups further differentiates the Ox receptor pharmacophore from the I₂-receptor pharmacophore in that compounds containing an imidazoline ring (where X is NH) have poor (μM) affinity at the Ox receptor binding site and high (nM) affinity at the I₂-RBS. While compounds containing an oxazoline ring (X is O) have high affinity (nM) for the Ox receptor binding site and poor (μM) affinity at the I₂-RBS. The Ox receptor pharmacophore can be further distinguished from the α₂-adrenoceptor pharmacophore by the substituents R₅ and R₆ as present in formula III. Substitutions in this region of the compound reduce, or abolish, α₂-adrenoceptor affinity while maintaining affinity for the Ox receptor.

While it is possible that, for use in therapy, compounds of the invention may be administered as the raw chemical, it is preferable to present the active ingredient as a composition. The composition may be a pharmaceutical composition.

The invention thus further provides pharmaceutical compositions comprising a compound of the invention or a pharmaceutically acceptable salt or derivative thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof.

For some formulations it may be preferable to prevent the active compound in the form of a pharmaceutically acceptable salt or ester.

Examples of pharmaceutically acceptable salts include inorganic and organic acid addition salts such as the hydrochloride, hydrobromide, phosphate, nitrate, perchlorate, sulphate, citrate, lactate, tartrate, maleate, fumarate, mandelate, benzoate, ascorbate, cinnamate, benzenesulfonate, methanesulfonate, stearate, succinate, glutamate, glycollate, toluene-p-sulphonate, formate, malonate, naphthalene-2-sulphonate, salicylate and the acetate. Such salts, and pharmaceutically acceptable esters, are formed by procedures well known in the art.

Other acids such as oxalic acid, while not in themselves pharmaceutically acceptable, may be useful in the preparation of salts useful as intermediates in obtaining compounds of the invention and their pharmaceutically acceptable acid addition salts.

Pharmaceutical compositions include those suitable for oral, rectal, nasal, topical (including buccal and sub-lingual), vaginal or parenteral (including intramuscular, sub-cutaneous and intravenous) administration or in a form suitable for administration by inhalation or insufflation.

The compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration, or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing one (1) milligrams of active ingredient or, more broadly, 0.001 to three hundred (300) milligrams, per tablet, are accordingly suitable representative unit dosage forms.

The compounds of the present invention can be administered in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “pharmaceutical composition” is intended to include the formulation of the active compound with encapsulating material as carrier providing a capsule in which the active component, with or without carriers, is surrounded by a carrier, which is thus in association with it.

Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The compounds according to the present invention may thus be formulated for parenteral administration (e.g. by injection, for example bolus injection or continuous infusion) and may be presented in unit dose form in ampoules, prefilled syringes, small volume infusion or in multi-dose containers with an added preservative. The compositions may take such forms as suspensions, solutions, or emulsions in oily or aqueous vehicles, and may contain formulatory agents such as suspending, stabilising and/or dispersing agents. Alternatively, the active ingredient may be in powder form, obtained by aseptic isolation of sterile solid or by Iyophilisation from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomising spray pump.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 5 microns or less. Such a particle size may be obtained by means known in the art, for example by micronization.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

A further aspect of the present invention contemplates an isolated Ox receptor polypeptide.

Preferably, the isolated Ox receptor polypeptide is in a sequencably pure form.

By “sequencably pure” is meant that the purified Ox receptor is provided in a form which is sufficiently homogeneous and, if necessary, modified to facilitate amino acid sequence determination.

The isolated Ox receptor may be characterised by a high binding affinity for O501 and a poor binding affinity for methoxyidazoxan, clonidine and idazoxan. Preferably the receptor is characterised by having a binding affinity for O501 of 1 to 500 nM and affinities for methoxyidazoxan, clonidine and idazoxan of greater than 1000. More preferably the receptor is characterised by having a binding affinity for O501 is between 10 and 100 nM and affinities for methoxyidazoxan, clonidine and idazoxan is greater than 5000 nM.

Standard methods may be used to isolate the Ox receptor polypeptide for example gel filtration, ion-exchange chromatography, hydrophobic interaction chromatography, reverse-phase chromatography, electrophoresis, affinity chromatography or immuno-affinity chromatography, amongst others or any combination thereof. Such methods are well-known to those skilled in the art and the use of all such methods to isolate an Ox receptor polypeptide are encompassed by the present invention.

In one preferred embodiment, the receptor can be identified by expression cloning by screening of commercially available cDNA libraries expressed in bacterial or mammalian cells. Preferred sources of these libraries are rat hippocampus and rat kidney. Positive clones expressing the Ox receptor are identified by screening for binding of tritiated rilmenidine or other suitably labelled oxazoline, in the presence of sufficient idazoxan (1 μM), moxonidine (220 nM) and 2-BFI (220 nM) to inhibit binding to the alpha₂, I₁ and I₂ receptors. Positive clones are selected and rescreened until single positive clones can be identified. cDNA from these clones is isolated and sequenced using standard techniques. The resulting cDNA sequence can be used to infer the corresponding amino acid sequence for the receptor.

In another preferred embodiment, the Ox receptor polypeptide is isolated using at least one affinity chromatography step, optionally in association with one or more other procedures for protein purification. As will be known to those skilled in the art, the isolation of an Ox receptor polypeptide using affinity chromatography requires a biospecific ligand capable of specifically binding to the intact Ox receptor or an Ox receptor polypeptide to be attached to a chromatographic matrix for example Sepharose™, Superose™, cellulose or Sephacryl™ (Pharmacia Fine Chemicals, Uppsala, Sweden), amongst others such that the inmmobilised ligand retains its specific binding affinity for the Ox receptor or Ox receptor polypeptide.

Preferably, the biospecific ligand is a chemical compound which exhibits specific and reversible binding affinity for the intact Ox receptor or an Ox receptor polypeptide.

Particularly preferred biospecific ligands contemplated by the invention include Ox receptor substrate compounds and substrate analogues, such as those described herein. More preferably, the biospecific ligand is a compound having the structure set forth in formula II, even more preferably a compound having the structure set forth in Formula II, which allows it to be attached to the chromatographic matrix.

The biospecific ligand is attached to the chromatographic matrix either directly or alternatively, via a spacer arm placed therebetween, for example a spacer arm comprising a short alkyl group having between 1 and 12 carbon atoms, to reduce steric interference between the matrix and the Ox receptor. Standard covalent coupling procedures may be employed, for example coupling via cyanogen bromide, carbodiimide, epoxy, thiopropyl, diazonium or bromoacetamidoalkyl groups, amongst others.

The Ox receptor in cellular extract or a derivative fraction thereof in a suitable “binding buffer”, is applied to the affinity chromatography column under conditions sufficient to promote binding of the receptor to the column. The non-specific binding of molecules other than Ox receptors may be minimised by including in the binding buffer one or more chemical compounds for which said molecules have a high binding affinity compared to the Ox receptor. Alternatively, or in addition, the binding buffer may include one or more protease inhibitors, serine protease inhibitors, trypsin inhibitors, leupeptin or PMSF, to minimise proteolysis of the Ox receptor during the isolation step. Once the Ox receptor is bound to the column, the column is washed several times with binding buffer to remove contaminants.

The bound Ox receptor may be eluted from the affinity matrix using a variety of elution methods known to those skilled in the art. Elution methods may be either selective or non-selective in nature. Usually, selective elution methods are applied in combination with group specific adsorbents and non-selective elution methods are used in combination with highly specific adsorbents. A compromise may have to be made between the harshness of the eluent required for elution and the risk of denaturing the eluted material. Forces which maintain the complex include electrostatic interactions, hydrophobic effects and hydrogen-bonding. Agents which weaken these interactions may be expected to function as efficient eluting agents. Elution may be achieved by either a step-wise or a continuous change in conditions. Continuous gradients are useful for their zone sharpening effect; the trailing edge of a desorbed substance experiences a higher concentration of eluting agent than the peak and is therefore made to move faster down the column than the main peak. When substances are very tightly bound to the absorbent, it may be effective to stop the flow after applying eluent and to allow the adsorbent to remain in the presence of eluting agent (30 min to 2 h is commonly used) before commencing elution. This allows dissociation to take place prior to elution and thus ensures good recoveries of adsorbed substances.

Changes in pH, ionic strength, polarity or the use of denaturing agents such as chaotropic salts, guanidine-HCl or urea or electrophoretic techniques may be used to desorb the bound Ox receptor or Ox receptor polypeptide from the affinity matrix.

Alternatively, the Ox receptor or Ox receptor polypeptide may be desorbed using a selective eluent capable of competing for the binding of the receptor or receptor polypeptide to the ligand. The selective eluent may be applied to the column in a suitable buffer composition at a single concentration or a concentration gradient of the eluent in the buffer may be used.

In a particularly preferred embodiment the Ox receptor or Ox receptor polypeptide is eluted using a selective eluent comprising O501.

The isolated Ox receptor polypeptide is provided in a sequencably pure form by any means of preparing proteins for amino acid sequence determination known to those skilled in the art. Preferably, the polypeptide is purified using at least one chromatographic or electrophoretic step.

Whilst the N-terminus of the isolated Ox receptor polypeptide may be sequenced, it is particularly desirable to obtain internal amino acid sequences of the Ox receptor polypeptide. Accordingly, a sequencably pure Ox receptor polypeptide will preferably comprise a proteolytic digestion product of the complete Ox receptor polypeptide, obtained for example by digestion of the isolated polypeptide with trypsin and/or endo-lysC. As with the isolated protein, the peptides obtained by proteolytic digestion are further purified to facilitate their amino acid sequence determination.

Peptides are generally transferred to PVDF membranes to facilitate the sequence determination of nanomolar quantities or even femtomolar quantities of peptide, and sequenced using Edman degradation or other techniques known to those skilled in the art.

The amino acid sequence of the Ox receptor polypeptide is useful for the preparation of degenerate pools of synthetic oligonucleotides encoding same or complementary thereto.

Those skilled in the art will be aware that such oligonucleotide pools are particularly useful as hybridisation probes and/or amplification primers in the isolation of nucleic acid molecules which encode the Ox receptor polypeptide described herein. In preparing oligonucleotide pools for such purposes, it is desirable to utilise those amino acid sequences of peptides which include one or more amino acid selected from the list comprising phenylalanine, tryptophan, tyrosine, cysteine, histidine, glutamine, asparagine, lysine, aspartate or glutamate or alternatively, which include no more than one amino acid selected from the list comprising leucine, serine and arginine. Selection of amino acid sequences in accordance with these parameters reduced the number of different oligonucleotide sequences which are required to provide a representative pool including all possible Ox receptor polypeptide-encoding sequences.

The oligonucleotides described herein will at least comprise a combination of any of the nucleotides adenine, cytidine, guanine, thymidine, or inosine, or functional analogues or derivatives thereof which are at least capable of being incorporated into a polynucleotide molecule without having an inhibitory effect on their use as hybridisation probes or primer molecules.

Furthermore, one or both of the nucleic acid primer molecules may be contained in an aqueous mixture of other nucleic acid primer molecules, for example a mixture of degenerate primer sequences which vary from each other by one or more nucleotide substitutions or deletions. Alternatively, one or both of the nucleic acid primer molecules may be in a substantially pure form.

The oligonucleotide sequences derived from isolated Ox receptor peptide sequences are employed to identify and isolate Ox receptor-encoding nucleotide sequences from cells, tissues, or organ types of any mammalian species which expresses an Ox receptor, in particular a laboratory mammal such as a rat, mouse or rabbit.

In one approach, genomic DNA, or mRNA, or cDNA derived from the cells, tissues or organs of said mammal, in particular kidney tissue, is contacted with a hybridisation effective amount of one or more oligonucleotide pools described herein or a complementary sequence and then the hybridising genomic DNA, or mRNA, or cDNA is detected.

In a particularly preferred embodiment, the Ox receptor-encoding oligonucleotide is end-labelled with a reporter molecule capable of giving an identifiable signal (e.g. a radioisotope such as ³²P or ³⁵S or a biotinylated molecule).

The hybridising genetic sequence thus identified is preferably in a recombinant form, in a virus particle, bacteriophage particle, yeast cell, animal cell, or a plant cell.

An alternative method contemplated in the present invention involves hybridising two nucleic acid “primer molecules” derived from the Ox receptor peptide amino acid sequence described herein to a nucleic acid “template molecule” comprising an Ox receptor-encoding gene or a part thereof. According to this embodiment, specific nucleic acid molecule copies of the template molecule are amplified enzymatically in a polymerase chain reaction, a technique that is well known to one skilled in the art.

In a preferred embodiment, each nucleic acid primer molecule is at least 10 nucleotides in length, more preferably at least 20 nucleotides in length, even more preferably at least 30 nucleotides in length, still more preferably at least 40 nucleotides in length and even still more preferably at least 50 nucleotides in length.

The nucleic acid template molecule may be in a recombinant form, in a virus particle, bacteriophage particle, yeast cell, animal cell, or a plant cell. Preferably, the nucleic acid template molecule is derived from a human or laboratory animal species.

Those skilled in the art will be aware that there are many known variations of the basic polymerase chain reaction procedure (McPherson et al, 1991). The present invention extends to the use of all such variations in the isolation of Ox receptor-encoding genetic sequences.

The isolated nucleic acid molecule which encodes the Ox receptor polypeptide or a functional part thereof may be cloned into a plasmid or bacteriophage molecule, such as for the production of recombinant gene products. Methods for the production of such recombinant plasmids, cosmids, bacteriophage molecules or other recombinant molecules are well-known to those of ordinary skill in the art and can be accomplished without undue experimentation. Accordingly, the invention further extends to any recombinant plasmid, bacteriophage, cosmid or other recombinant molecule comprising the nucleic acid molecules described herein.

Still yet another aspect of the invention contemplates a method of identifying a modulator of Ox receptor activity, said method comprising assaying recombinant Ox receptor activity in the presence of a potential modulator and comparing said activity to the activity of recombinant Ox receptor in the absence of said potential modulator.

As used herein, the term “modulator” shall be taken to refer to any chemical compound, molecule or macromolecule which is capable of altering the activity of an Ox receptor polypeptide, in particular a mammalian Ox receptor polypeptide, including both agonists and antagonists of said activity.

Preferably, the subject method further comprises the first step of expressing a functional recombinant Ox receptor polypeptide in a cell for a time and under conditions sufficient for said polypeptide to be produced in an assayable quantity.

The term “assayable quantity” refers to a level of expression of a recombinant polypeptide which is sufficient for the activity of said polypeptide to be determined by any standard assay procedure which is specific for the function of the recombinant polypeptide.

A further aspect of the invention contemplates a modulator of Ox receptor activity, such as a competitive or non-competitive inhibitor, a competitive or non-competitive activator molecule or a molecule which binds irreversibly to an Ox receptor without leading to, activation thereof or alternatively, an antibody molecule or catalytic antibody molecule capable of binding and inhibiting the activity of a Ox receptor polypeptide, amongst others.

Preferably, the modulator is one which is identified using the method described supra for the identification of modulators of Ox receptor activity.

The modulator molecules described herein are useful in a wide range of prophylactic and therapeutic applications, by virtue of their ability to agonise or antagonise Ox receptor activity. Antagonists are particularly useful in the treatment of physiological or medical conditions wherein Ox receptor activity is elevated or it is desirable to reduce Ox receptor activity, whilst agonists are useful in the treatment of any conditions associated with reduced Ox receptor activity or where it is desirable to increase Ox receptor activity.

In order to facilitate an understanding of the invention reference will be made to the following examples which illustrate some preferred embodiments of the invention and demonstrate that the Ox receptor is a new receptor, distinct from I₂ receptors and α₂-adrenoceptors. However it is to be understood that the particularity of the following description is not to supersede the generality of the preceding description of the invention.

### EXAMPLE 1

a) 1-Dicyclohexylmethylamino-3-chloroethyl urea: Chloroethyl isocyanate (11 g in 16 ml of tetrahydrofuran) is added dropwise into a solution of dicyclohexylmethylamine (19.5 g in 125 ml of tetrahydrofuran). After 2 hrs the precipitated solid is filtered, washed with tetrahydrofuran and air-dried. Yield=23.4 g, 78%.

b) 2-(Dicyclohexylmethylamino)-oxazoline) (0503): 1 -Dicyclohexylmethylamino-3-chloroethyl urea, 36.1 g, finely powdered is suspended in 75 ml water. Sufficient tetrahydrofuran is then added to dissolve the solid followed by a 10% solution of aqueous sodium hydroxide (26 ml). After refluxing for 1 hr the solvent is removed under reduced pressure. The residue is extracted with ethyl acetate. The extracts are washed with brine, dried over sodium sulfate and evaporated to dryness. The residue is recrystallised from ethyl acetate:hexane. m.p.=135-138° C. Yield=9.45 g, 59%. Proton nmr spectrum in deuterated chloroform shows δ 0.6-2.0 (22H broad doublet, cyclohexyl protons), δ 3.2-4.2 (5H m, oxazoline and methine protons), δ 4.7 (1H s, NH). Chemical ionisation mass spectrum shows MH+ ion at m/e 265.

### EXAMPLE 2

a) (R-(−)-1-(1-Cyclohexylethyl)-3-chloroethyl urea: Chloroethyl isocyanate (11 g in 16 ml of tetrahydrofuran) is added dropwise into a solution of (R)-(−)-1-(1-cyclohexylethylamine (12.7 g in 125 ml of tetrahydrofuran). After 2 hrs the precipitated solid is filtered, washed with tetrahydrofuran and air-dried. Yield=15.3 g, 75%. m.p.=109-113° C.

b) (R-(−)-2-(1-Cyclohexylethylamino)-oxazoline) ((R)-(−)-O504): (R)-(−)-1-(1-Cyclohexylethyl)-3-chloroethyl urea, 13.7 g, finely powdered is suspended in 75 ml water. Sufficient tetrahydrofuran is then added to dissolve the solid followed by a 10% solution of aqueous sodium hydroxide (26 ml). After refluxing for 1 hr the solvent is removed under reduced pressure. The residue is extracted with ethyl acetate. The extracts are washed with brine, dried over sodium sulfate and evaporated to dryness. The residue is recrystallised from ethyl acetate:hexane, m.p.=76-79° C. Yield=7.6 g, 58%. Proton nmr spectrum in deuterated chloroform shows 80.9-1.9 (11H bd, cyclohexyl protons), δ 1.07 (3H d, methyl protons), δ 3.5-4.3 (4H m, oxazoline protons), δ 3.4 (1H q, methine proton), δ 4.5 (1H s, NH). Chemical ionisation mass spectrum shows MH+ ion at m/e 197.

### EXAMPLE 3

a) (S)-(+)-1-(1-Cyclohexylethyl)-3-chloroethyl urea: Chloroethyl isocyanate (11 g in 16 ml of tetrahydrofuran) is added dropwise into a solution of (S)-(+)-1-(1-cyclohexylethylamine (12.7 g in 125 ml of tetrahydrofuran). After 2 hrs the precipitated solid is filtered, washed with tetrahydrofuran and air-dried. Yield=15.5 g, 76%, m.p.=107-110° C.

b) (S)-(+)-2-(1-Cyclohexylethylamino)-oxazoline ((S)-(+)-O504): (S)-(+)-1-(1-Cyclohexylethyl)-3-chloroethyl urea, 13.7 g, finely powdered is suspended in 75 ml water. Sufficient tetrahydrofuran is then added to dissolve the solid followed by a 10% solution of aqueous sodium hydroxide (26 ml). After refluxing for 1 hr the solvent is removed under reduced pressure. The residue is extracted with ethyl acetate. The extracts are washed with brine, dried over sodium sulfate and evaporated to dryness. The residue is recrystallised from ethyl acetate:hexane, m.p.=70-75° C. Yield=7.4 g, 56%. Proton rnir spectrum in deuterated chloroform shows δ 0.9-2.0 (11H bd, cyclohexyl protons), δ 1.1 (3H d, methyl protons), δ 3.5-4.2 (4H m, oxazoline protons), δ 3.4 (1H q, methine proton), δ 4.5 (1H s, NH). Chemical ionisation mass spectrum shows MH+ ion at m/e 197.

### EXAMPLE 4

a) (R)-2-(1-(4-nitrophenyl))ethylaminooxazoline (R—O505NO₂). (R)-alphamethyl-4-nitrobenzylamine hydrochloride (645 mg, 0.32 mmole) was suspended in 5 ml tetrahydrofuran and stirred with triethylamine (330 mg) for 1 hour. 2-chloroethylisocyanate (360 mg, 95%) was added in 5 ml tetrahydrofuran with stirring and the mixture stirred for 4 hours. 6 ml of water was then added followed by 2 ml 10 molar sodium hydroxide and the mixture heated to reflux for 24 hours. The mixture was then diluted with 50 ml ethyl acetate and shaken. The aqueous phase was discarded and the organic layer washed with 5 ml water, then extracted twice with 5 ml 1 molar hydrochloric acid. The extracts were combined and basified with 10 molar sodium hydroxide. The precipitated product was extracted with 25 ml ethyl acetate, washed with water and then reduced to dryness in vacuo. The product forms pale yellow crystals on drying, mp. 112-116° C. Yield 0.62 g (83%). Proton nmr in CDCl₃ shows 7.8 (4H dd, J=9 and 42 Hz), 4.8 (1Hq, J=7 Hz), 3.9 (4Hm) 1.4(3H d, J=7 Hz).

b) (R)-2-(1-(4aminophenyl))ethylaminooxazoline (R—O505NH₂). (R)-2-(1-(4-nitrophenyl))ethylaminooxazoline (600 mg) was dissolved in 5 ml 1 molar hydrochloric acid and stirred with powdered tin (400 mg) After 2 hours the solution was basified with sodium hydroxide and extracted with ethyl acetate (50 ml). The extract was washed twice with water (10 ml) and reduced to dryness in vacuo. The product was a colourless oil 500 mg. Proton nmr in CDCl₃, 6.8 (4Hq), 4.6 (1H q, J=7 Hz) 3.9(4H m), 1.4 (3Hd, J=7 Hz).

c) 2-(4-hydroxyphenyl)ethylaminooxazoline. (0508) Tyramine hydrochloride (1.04 g) was stirred in 5 ml tetrahydrofuran with triethylamine 860 μl for 16 hours, after which 2-chloroethylisocyanate (540 μl) was added and stirring continued for 24 hours. The mixture was extracted into ethyl acetate and washed with 1M hydrochloric acid (10 ml) and water, before reducing to dryness in vacuo. The residue was redissolved in tetrahydrofuran (5 ml) and water (5 ml) containing sodium sulfite (50 mg). 5 molar sodium hydroxide (2 ml) was added and the mixture heated to reflux for 5 hours. Sodium dihydrogen phosphate (1 g was then added with stirring) and the mixture reduced to dryness in vacuo. The product was isolated by extraction with chloroform (10 ml) and isopropanol (2×10 ml). The combined extracts were reduced to dryness and reconstituted in ethyl acetate containing 10% methanol. The mixture was separated by chromatography on silica gel, and eluted with mixtures of ethyl acetate and methanol. The combined eluates were reduced to dryness to give a crystalline solid (450 mg, 35%), mp. 90-96° C. Proton nmr in deuterated methanol shows, 6.7 (4H q), 4.5- 3.0 (6H m) 2.5 (2H br.tr., J=8 Hz). Chemical ionisation mass spectrum shows m/e 207 (MH+).

d) 2-(4-benzyloxyphenyl)propylaminooxazoline. (O509) 4-benzyloxyphenylethylamine 1.256 g was dissolved in 10 ml tetrahydrofuran, 2-chloroethylisocyanate (0.55 g) was added and stirred for 16 hours. 5 ml % molar sodium hydroxide was then added and the mixture heated to reflux for 5 hours. On cooling, ethyl acetate 50 ml was added and the aqueous fraction discarded. The organic phase was washed twice with water (10 ml) then extracted with 10 ml 1 molar hydrochloric acid. The extract was basified with sodium hydroxide, and the precipitated solid extracted with ethyl acetate (50 ml). The extracts were washed with water then reduced to dryness in vacuo. Yield 800 mg (50%) of a white crystalline solid, mp 34-47° C. Proton nmr in CDCl₃. 7.3 (5Hs), 6.8 (4H q,), 4.9 (2Hs), 3.9 (6H br m), 2.6 (2H br.d., J=6 Hz), 1.05 (3H tr, J=7 Hz). Chemical ionisation mass spectrum shows m/e 311 (MH+).

e) 2-phenoxyethylaminooxazoline (O510) 2-phenoxyethylamine (0.714 g) was dissolved in 10 ml tetrahydrofuran and 2-chloroethylisocyanate (0.55 g) was added with stirring. After 4 hours at room temperature 2 ml of 10% sodium hydroxide was added and the mixture heated to reflux for 5 hours. The solution was extracted into ethyl acetate (50 ml), washed with water (10 ml), then extracted into 10 ml 1 molar hydrochloric acid. This extract was basified with sodium hydroxide and reextracted into ethyl acetate (25 ml). The extract was washed with water and reduced to dryness leaving a white waxy crystalline solid. Proton nmr in CDCl₃ δ7.0 (5H m), δ3.4-4.4 (8H m). Chemical ionisation mass spectrum shows m/e 207 (MH+).

f) 2-(4-methoxyphenoxy)ethylaminooxazoline (O511) 2-(4-methoxyphenoxy)ethylamine (0.871 g) was dissolved in 10 ml tetrahydrofuran and 2-chloroethylisocyanate (0.55 g) was added with stirring. After 4 hours at room temperature 2 ml of 10% sodium hydroxide was added and the mixture heated to reflux for 5 hours. The solution~ was extracted into ethyl-acetate (50 ml), washed with water (10 ml), then extracted into 10 ml 1 molar hydrochloric acid. This extract was basified with sodium hydroxide and reextracted into ethyl acetate (25 ml). The extract was washed with water and reduced to dryness leaving a white waxy crystalline solid. Proton nmr in CDCl₃ δ6.9 (4H s), δ3.4-4.4 (8H m) δ3.8 (3H s). Chemical ionisation mass spectrum shows m/e 237 (MH+).

The compounds prepared above are represented in Table 1 below together with selectivity data where available.

TABLE 1

| | | R7, | | | | Selectivity |
|---|---|---|---|---|---|---|
| Compound | Formula | R8 | Z | Φ | R12 | Ox/apha2 |
| (R)-O505-NO₂ | X | H | NH | 4-nitrophenyl | CH₃ | |
| (R)-O505-NH₂ | X | H | NH | 4-aminophenyl | CH₃ | |
| O508 | VIII | H | CH₂CH₂NH | 4-hydroxyphenyl | | 100 |
| O509 | VIII | H | CH₂CH₂NH | 4-benzyloxyphenyl | | 2.0 |
| O510 | VIII | H | CH₂CH₂NH | phenoxy | | |
| O511 | VIII | H | CH₂CH₂NH | 4-methoxyphenoxy | | |

### EXAMPLE 5

Receptor Affinities and Selectivities of Selected Drugs for Ox receptors, α2-adrenoceptors and I₂-receptors in Rat Brain Homogenates

The following compounds were tested for their affinities and selectivities for Ox, I₂ and α₂-receptors in brain homogenates.

TABLE 2

| Compound | Formula* | R₁ | R₂ | R₃ | R₄ | R₁₀ | R₁₁ | R₁₂ |
|---|---|---|---|---|---|---|---|---|
| Rilmenidine | II | cyclopropyl | cyclopropyl | — | — | — | — | — |
| BAY-A6781 | III | — | — | methyl | ethyl | — | — | — |
| (+)-S8349 | II | CF₃ | cyclopropyl | — | — | — | — | — |
| (−)-S8350 | II | CF₃ | cyclopropyl | — | — | — | — | — |
| O501 | III | — | — | ethyl | ethyl | — | — | — |
| O502 | II | phenyl | phenyl | — | — | — | — | — |
| O503 | VI | — | — | — | — | H | H | — |
| (S)-(+)-O504 | VII | — | — | — | — | — | — | methyl |
| (R)-(−)O504 | VII | — | — | — | — | — | — | methyl |
| (S)-(−)-O506 | II | methyl | l-naphthyl | — | — | — | — | — |

*where X is O, Y is —CH₂CH₂—, Z is NH and, for formula II, W is —CHR¹R², for formula III R⁵ and R⁶ are hydrogen, and for formulae III, VI and VII R⁷ and R⁸ are H.

Methoxyidazoxan is 2-(2-methoxy- 1,4-benzodioxan)-imidazoline); clonidine is 2-(2,6-dichlorophenylamino)-imidazoline; idazoxan is 2-(1,4-benzodioxan)-imidazoline; cirazoline is 2-(2-cyclopropylphenoxymethyl)-imidazoline; BU-224 is 2-(4,5-dihydroimidaz-2-yl)quinoline.

The results are shown below in Tables 3 and 4.

TABLE 3

| | Receptor Affinities (nM) | | |
|---|---|---|---|
| Compound | Ox | I₂ | Selectivity Ox/I₂* |
| Methoxyidazoxan | 319,000 | 24,400 | 0.08 |
| Clonidine | 7030 | 5270 | 0.7 |
| Idazoxan | 62,300 | 2.3 | 0.00004 |
| Cirazoline | 21,200 | 1.6 | 0.00008 |
| BU-224 | >100,000 | 0.9 | <0.000009 |
| Rilmenidine | 15 | 36 | 2.4 |
| O501 | 18 | 6,900 | 383 |
| O502 | 160 | 1,900 | 12 |
| O503 | 117 | 3,800 | 32 |
| (S)-(+)-O504 | 106 | 707 | 6.7 |
| (R)-(−)-O504 | 31 | 56 | 1.8 |

*Compounds more selective for the Ox site have a selectivity > 1

TABLE 4

| | Receptor Affinities (nM) | | |
|---|---|---|---|
| Compound | Ox | α₂ | Selectivity Ox/α₂* |
| Methoxyidazoxan | 319,000 | 1.9 | 0.000006 |
| Clonidine | 7030 | 2.7 | 0.0004 |
| Idazoxan | 62,300 | 15.6 | 0.0003 |
| Cirazoline | 21,200 | 2.6 | 0.0001 |
| BU-224 | >100,000 | 5,477 | <0.1 |
| Rilmenidine | 15 | 410 | 27 |
| O501 | 18 | 210 | 12 |
| O502 | 160 | 380 | 2.4 |
| O503 | 117 | 640 | 5.5 |
| (S)-(+)-O504 | 20 | 30 | 1.5 |
| (R)-(−)-O504 | 32 | 590 | 18 |

*Compounds more selective for the Ox site have selectivity of > 1

The affinity and selectivity of the claimed compounds in the rat is exemplified by comparisons with conventional ligands in Tables 3 and 4, in which selectivity is defined as the reciprocal of the ratio of the affinities in nanomolar concentrations for each site.

In the rat, selective α₂-adrenoceptor antagonists such as methoxyidazoxan exhibit poor affinity (μM) for both the Ox receptor and the I₂-receptor and high affinity (nM) for the α₂-adrenoceptor. Such a binding pattern indicates that the Ox receptor and I₂-receptors are distinct sites from the a₂-adrenoceptors. Clonidine, which has been reported widely to be an I₁ and α₂-receptor ligand, also displays poor affinity (μM) for both the Ox receptor and I₂-receptor and high affinity (μM) for α₂-adrenoceptors, indicating that both Ox receptors and I₂-receptors are binding sites distinct from the I₁- and α2-receptors. Known I₂-receptor ligands, such as idazoxan, cirazoline and BU-224, display poor (μM) affinity for the Ox receptor and high affinity (nM) for the I₂-receptor, consistent with being highly selective I₂ ligands. This indicates that the Ox receptor and I₂-receptor are distinct. The I₂-receptor ligands idazoxan and cirazoline also have a high affinity (nM) for α₂-adrenoceptors, whereas BU-224 has poor affinity (μM) for this receptor. The oxazoline rilmenidine is a known α₂-adrenoceptor agonist which has a high affinity (nM) for both the Ox receptor and the I₂-receptor in the rat but lower affinity in human brains.

### EXAMPLE 6

Receptor Affinities and Selectivities of Selected Drugs for Ox receptors and I₂-Receptors in Human Brain Homogenates.

The following compounds were also tested for their affinities and selectivities for Ox receptors and I2- receptors in human brain homogenates. The results are shown in Table 5.

TABLE 5

| | Receptor Affinities (nM) | | |
|---|---|---|---|
| Compound | Ox | I₂ | Selectivity Ox/I₂ |
| Methoxyidazoxan | 100,000 | >100,000 | 1 |
| Clonidine | 17,930 | 18,460 | 1.0 |
| Idazoxan | 6060 | 4.8 | 0.0008 |
| Cirazoline | 1074 | 1.6 | 0.0015 |
| BU-224 | 9576 | 2.2 | 0.0002 |
| BAY-A6781 | 215 | 8,250 | 38 |
| Rilmenidine | 396 | 5,220 | 13 |
| (+)-S8349 | 268 | 13,000 | 49 |
| (−)-S8350 | 199 | 7,700 | 32 |
| O501 | 38 | 9,100 | 239 |
| O502 | 234 | 686 | 3 |
| O503 | 70 | 4,800 | 69 |
| (S)-(+)-O504 | 180 | 2,190 | 12 |
| (R)-(−)-O504 | 300 | 2,700 | 9 |
| (S)-(−)-O506 | 99 | 7,580 | 77 |

*Compounds with selectivity > 1 show significant selectivity for the Ox receptor.

In humans, all of the oxazolines tested displayed significant selectivity (3-239) for the Ox receptor over the I₂-receptor.

Measurement of the affinity and selectivity of such compounds for the a₂-adrenoceptor, I₂-receptor and Ox receptor binding sites may be carried out by either of two procedures, i.e. ligand displacement studies in membranes of brain and kidney or by autoradiography of brain and kidney sections. In both cases the radioligand is tritiated rilmenidine ([³H]-RIL). Rilmenidine is an oxazoline compound, which is an effective anti-hypertensive agent and originally believed to act as a central and peripheral α₂-adrenoceptor agonist. As rilmenidine not only binds to α₂-adrenoceptor sites but also in the rat to I-receptors, Ox receptor sites may be preferentially labelled by incubation with [³H]-RIL in the presence of μM concentrations of α₂-adrenoceptor ligands such as adrenaline or methoxyidazoxan with or without the addition of the I₁- and I₂-receptor ligands clonidine and idazoxan. Analysis of the displacement of [³H]-RIL from these sites by drugs reveals the affinity of the drug for each binding site.

Receptor Binding Studies

Brain Membrane Preparation

Male WKY rats are stunned and decapitated. The brains are removed and the forebrain dissected free, then frozen over liquid nitrogen before being stored at −70° C. Human brain cortex samples are obtained within 28 hours of death and frozen over liquid nitrogen before being stored at −70° C. On the day of the measurement the cortex (human or rat) is homogenised in 20 vol ice-cold 50 mM Tris.HCl buffer (pH 7.4) using a Polytron tissue homogeniser. The homogenate is centrifuged at 37000×g for 10 min and the pellet resuspended in 20 vol of fresh buffer and recentrifuged. The pellet is then resuspended in 33 vol of 50 mM Tris.HCl buffer for use in binding assays. Where adrenaline is used in binding assays the buffer also contains 25 μM EDTA and 10 μM pargyline.

Measurement of Ox Receptor Binding Sites

Drug inhibition of [³H]-RIL (38-49 Ci/mmol) binding is measured in duplicate in samples containing 5 nM [³H]-RIL and competing drug 0.01 nM to 10 mM, 10 μM adrenaline and 15 mg of membranes in a final volume of 1 ml. After incubation for 30 min at 25° C., the membranes are recovered by filtration onto Whatman GF/B glass fibre filters presoaked in 1.0% polyethyleneimine. The filters are washed 3 times with cold assay buffer before measurement of bound radioactivity by scintillation counting. The binding data are analysed by an iterative non-linear computerised curve fitting program “RADLIG” version 4.0 (McPherson, 1983).

Measurement of α₂-Adrenoceptor and I₂-Receptor Binding Sites

Brain membranes prepared as above, are incubated in the presence of [³H]-methoxyidazoxan (52-57 Ci/mmol) 0.5 nM or [³H]-idazoxan (43-45Ci/mmol) 1.6 nM in Tris.HCl buffer pH 7.4 for α₂-adrenoceptor and I₂-receptor binding respectively. Inhibition of binding is measured in the presence of increasing amounts of test compound (0.1 nM to 1 mM). Non-specific binding is estimated by addition of 10 μM phentolamine or 100 μM cirazoline respectively. Membrane bound radioactivity is isolated and measured as above by vacuum filtration, washing and scintillation counting. Data is analysed by the “RADLIG” program.

Autoradiographic Studies

Preparation of Tissue Sections

Male WKY rats are stunned and decapitated. The brains, kidneys or spinal cords are rapidly excised, frozen over liquid nitrogen and stored at −70° C. until used. The tissues are then mounted onto microtome chucks with Tissue-Tek embedding matrix. Horizontal, coronal or sagittal sections are cut (10 to 14 μm thick) at −15° C. and thaw mounted onto cold gelatine/chrome alum coated slides. 2 to 4 sections are mounted on each slide, air-dried at room temperature, then stored with silica gel dessicant at −70° C. up to 2 weeks before use.

Measurement of Ox Receptor Binding Sites in Tissue Sections

The mounted sections are brought to room temperature and preincubated for 20 min in 50 mM Tris.HCl buffer (pH 7.6) to remove endogenous inhibitors such as noradrenaline. The slides are then dried at room temperature. Each section is then covered with the same buffer containing 40 nM [³H]-RIL±1 μM adrenaline or 10 μM of the oxazoline BAY A6781 and incubated at room temperature for 60 min to assess total, non-adrenergic and non-specific binding respectively. Breakdown of adrenaline is minimised by addition of 25 μM EDTA and 10 μM pargyline. The incubations are terminated by washing the sections in fresh buffer for 4 min and rinsing in distilled water. Sections are then air-dried and desiccated overnight. The sections are then apposed to tritium sensitive (Hyperfilm™-³H, Amersham) film in X-ray cassettes for 4 to 16 weeks at 4° C. After exposure the films are developed in Kodak D19 at 18 to 20° C. for 5 min. Autoradiograms are analysed by computer aided densitometry and calibrated by co-exposed tritium standards.

### EXAMPLE 7

Method for Isolating Novel Receptors:

Rat hippocampus and rat kidney cDNA libraries are available from Stratagene. The libraries are prepared in the Lambda ZAP® II vector cloning system.

Bacteriophage are plated at approximately 40,000 pfu/plate with the appropriate E. coli strain on 150 mm LB agar plates, using 7 ml of 1% top agar. The plates are incubated for 2-4 hours at 37° C. until plaques are pinpoint size. Protein expression is then induced by overlaying plaques with filters saturated with IPTG (isopropyl theoglactosidase) (soaked in 10 mM solution and dried at RT). Plates and filters are then incubated for a further 8-12 hours until plaques are approximately I mm in size.

After one hour at 4° C. the orientation of each filter is marked asymmetrically and lifted. Filters are then air dried for 5 minutes at RT. Plaques are overlayed with a duplicate set of IPTG-soaked filters, and then induced as for first set. Filters are blocked at room temperature for 30 minutes in 50 nM Tris pH 7.6 with 10 μM adrenaline and 100 nM idazoxan. 5 nM ³H-Rilmenidine or other radiolabelled ligand is added and this is incubated for 2-24 hours at 4° C. in a heat sealed bag on a rotating platform. Filters are washed 4-5 times in blocking buffer for 5-10 minutes at 4° C. Filters are then blot dried, and exposed to tritium sensitive x-ray film (Hyperfilm TM-3H, Amersham) at −70° C.

Positive phage clones are picked from the plates using the autoradiograms, and secondary/tertiary screens performed until an individual clone or clones are obtained.

The cloned DNA-phasemid is then released by co-infection with helper phage to generate sub-clones for plasmid DNA preparation and subsequent DNA sequencing, by dideoxynucleotide chain terminating sequencing.

### EXAMPLE 7

Method of Isolating and Sequencing the Oxazoline Receptor

A tissue rich in Ox receptors, such as rabbit kidney, is homogenised in 10 mM Tris buffer containing 5 mM EGTA, 5 mM EDTA and 10 μM phenylmethylsulfonyl fluoride pH7.4 at 4° C. The homogenate is subjected to differential centrifugation to isolate the plasma membrane fraction. The protein is solubilised with buffer containing octylglucoside detergent and purified by affinity chromatography with a column of sepharose coupled to a ligand such as formula II where X is O, Y is —CHR⁵CHR⁶— where R⁵ and R⁶ are H, W is —CHR¹R² wherein R₁ is 4-amino,2-methoxyphenyl, R₂ is H and Z is NH. Binding of α₂-adrenoceptors and I-receptors to the column can be minimised by the addition of methoxyidazoxan and 2-BFI, and the Ox-receptor selectively eluted with buffer containing O501. The eluate is then dialysed against drug-free buffer to remove O501. The Ox receptors in the eluate are selectively labelled with a photoaffinity label such as (¹²⁵I)-AZIPI (Lanier et al, 1995) or related compounds derived from formula II where X is O, Y is —CHR⁵CHR⁶— where R⁵ and R⁶ are H, W is —CHR¹R² wherein R¹ is 4-amino, 2-methoxyphenyl, R₂ is H and Z is NH; by exposure to ultraviolet light. The labelled Ox receptors are then precipitated with trichloroacetic acid, washed with ether and solubilised in Laemmli loading buffer and electrophoresed on 10% polyacrylamide gel under denaturing conditions. After electrophoresis, the gels are dried under vacuum and exposed to X-ray film for 1 to 8 days. The labelled bands can be located after development of the X-ray films and transferred electrophoretically to polyvinylidene difluoride membranes. The areas corresponding to the labelled Ox receptors are cut out and digested with trypsin. The generated peptides can then be separated by microbore reversed phase high performance liquid chromatography and the amino acid sequence of each determined. These sequences can then be used as the basis for generation of oligonucleotide probes for the dna sequence of the Ox receptor.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps of features.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

References

Alemany R, Olmos G, Escriba P V, Menargues A, Obach R and Garcia-Sevilla J A. LSL 60101, a selective ligand for imidazoline I-2 receptors, on glial fibrillary acidic protein concentration. Eur. J. Pharmacol. 1995; 280, 205-210.
Evans R G Current status of putative imidazoline (I₁) receptors and renal mechanisms in relation to their antihypertensive therapeutic potential. Clin. Exp. Pharmacol. Physiol. 1996; 23: 845-854.
Gustafson I, Westerberg, E. and Wieloch, T. Protection against ischemia-induced neuronal damage by α2-adrenoceptor antagonist idazoxan: influence of time of administration and possible mechanisms of action. J. Cereb. Blood Flow Metab. 1990; 10, 885-894.
Hosseini R, King P R, Louis W J and Gundlach A L. [³H]2-(2-benzofuranyl)-2-imidazoline, a highly selective radioligand for I₂-imidazoline receptor binding sites: Studies in rabbit kidney membranes. Naun.-Schmied. Arch. Pharmacol. 1997; 355:131-138.
King P R, Gundlach A L and Louis W J. Quantitative autoradiographic localization in rat brain of α₂-adrenoceptors and non-adrenergic I-receptor binding sites labelled by [3H] rilmenidine. Brain Res. 1995; 675, 264-278.
Lanier B, Raddatz R, Bakthavachalam V, Coupry I, Neumeyer J L and Lanier S M. Structural and Ligand Recognition Properties of Imidazoline Binding Proteins in Tissues of Rat and Rabbit. Mol. Pharmacol. 1995; 48: 703-710.
Lione L A, Nutt D J and Hudson L. [³H]2-(2-Benzofuranyl)-2-imidazoline: a new selective high affinity radioligand for the study of rabbit brain imidazoline I₂ receptors. Eur. J. Pharmacol. 1996; 304, 221-229.
Mallard N J, Hudson A L and Nutt D J Characterization and autoradiographical localization of non-adrenoceptor idazoxan binding sites in the rat brain. Br. J. Pharmacol. 1992; 106, 1019-1027.
McPherson, G A A practical computer-based approach to the analysis of radioligand binding experiments. Comput. Programs Biomed. 1983; 17: 107-113.
McPherson, M. J., Quirke, P. and Taylor, G. R. (1991) PCR A Practical Approach. IRL Press, Oxford University Press, Oxford, United Kingdom.
Nero T L, Hosseini A R, King P R, Iakovidis D, Gundlach A L and Louis W J. Development of an I₂-receptor pharmacophore, presented at the International Workshop on Imidazoline Receptors, Sep. 10, 1997, Melbourne, Victoria, Australia (Satellite Meeting to the International Society of Autonomic Neurosciences 1997), p44 of the conference handbook.
Olmos G, Alemany R, Escriba P V and Garcia-Sevilla J A. The effects of chronic imidazoline drug treatment on glial fibrillary protein concentrations in rat brain. Br. J. Pharmacol. 1994; 111, 997-1002.
Parini A, Moudanos C G, Pizzinat N and Lanier S M. The Elusive Family of Imidazoline Binding Sites. TIPS 1996; 17: 13-16.
Petito C K, Morgello S, Felix J C and Lesser M L. The two patterns of reactive astrocytosis in postischemic rat brain. J. Cereb. Blood Flow Metab. 1990: 10, 850-859.
Regunathan S, Feinstein D L and Reis D J. Expression of non-adrenergic imidazoline sites in rat cerebral cortical astrocytes. Neurosci. Res. 1993; 34: 681-688.
Regunathan S and Reis D J. Imidazoline receptors and their endogenous ligands. Annu. Rev. Pharmac. Toxicol. 1996; 36: 51-44.
Sastre M and Garcia-Sevilla J A. Opposite age-dependent changes of α_{2A}-adrenoceptors and [³H]Idazoxan binding sites (I₂-imidazoline sites) in the human brain: strong correlation of I₂ with MAO-B sites. J. Neurochem. 1993; 61: 881-889.
Tesson F, Limon-Boulez I, Urban P, Puype M, Vandekerckhove J, Coupry I, Pompou D and Parini A. Localisation of I₂-Imidazoline Binding Sites on Monoamine Oxidases. J. Biol. Chem. 1995; 270: 9856-9861.
Timmermans, PBMWM and van Zwieten P A. QSAR in centrally acting imidazolines structurally related to clonidine. J. Med. Chem. 1977; 20: 1636-1645.

1-44. (canceled)

45. A method for the treatment of diseases of the central nervous system (excluding those involving CNS depressant action), the cardiovascular system (excluding hypertension), the kidney, or diseases associated with abnormal adrenal gland secretions, or for the treatment of hyperglycaemia or peptic ulcer, which comprises administering an effective amount of a compound of formula II: 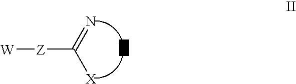
wherein W is optionally substituted aryl; —CHR¹R² where R¹ and R² are independently selected from hydrogen, optionally substituted C₁-C₆ alkyl, optionally substituted C₃-C₇ cycloalkyl and optionally substituted aryl or R¹ and R² are linked to form an optionally substituted C₅-C₇ cycloalkyl; OR′ where R′ is optionally substituted aryl; optionally substituted C₃-C₇ cycloalkyl; or optionally substituted C₁-C₆ alkyl; provided that R¹ and R² are not both hydrogen;
Z is imino, C₁-C₂ alkylene, —CH₂NH— or —CH₂CH₂NH—;
X is O or S; and
Y is optionally substituted C₂-C₃ alkylene; provided that W is not OR′ when Z is imino or —CH₂NH—;
or a pharmaceutically acceptable salt or ester thereof.

46. The method according to claim 45 wherein the disease is a disease of the central nervous system selected from the group consisting of dementia, mood disturbances, degenerative conditions and neurodegenerative diseases.

47. A method for the treatment of glaucoma comprising administering an effective amount of a compound of formula II 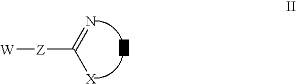
wherein W is optionally substituted aryl; —CHR¹R² where R¹ and R² are independently selected from hydrogen, optionally substituted C₁-C₆ alkyl, optionally substituted C₃-C₇ cycloalkyl and optionally substituted aryl or R¹ and R² are linked to form an optionally substituted C₅-C₇ cycloalkyl; OR′ where R′ is optionally substituted aryl; optionally substituted C₃-C₇ cycloalkyl; or optionally substituted C₁-C₆ alkyl; provided that R¹ and R² are not both hydrogen;
Z is imino, C₁-C₂ alkylene, —CH₂NH— or —CH₂CH₂NH—;
X is O or S; and
Y is optionally substituted C₂-C₃ alkylene; provided that W is not OR′ when Z is imino or —CH₂NH—; and
with the further provisos that
a) when Y is CH₂CH₂, X is O and Z is imino then
(i) if W is CHR¹R² and R¹ is H then R² is not selected from phenyl; phenyl substituted with methoxy, Br, Cl, F or trifluoromethyl; 3-nitrophenyl; 3- or 4-methylphenyl; 2- or 4-bromomethyl phenyl; 2- or 4-chloromethylphenyl; or 2,3- or 2,6-dimethylphenyl; and
(ii) if W is CHR¹R² and R¹ is CH₃ or cyclopropyl then R² is not phenyl or phenyl substituted with alkyl, halomethyl, fluoro or trifluoromethyl; and
b) when Y is (CH₂)₂₋₄, X is O or S, Z is imino and W is CHR¹R², then
(i) if R¹ is CF₃, CF₂CF₃ or CF₂CF₂CF₃ then R² is not alkyl, optionally substituted cycloalkyl or optionally substituted aryl, and
(ii) if R¹ is optionally substituted cyclopropyl, R² is not H, alkyl or optionally substituted cyclopropyl;
or a pharmaceutically acceptable ester or salt thereof, to a subject in need thereof.

48. A method for the treatment of diseases of the central nervous system, cardiovascular system, or the kidney, or for the treatment of diseases associated with abnormal adrenal gland secretions, or in the treatment of hyperglycaemia, glaucoma, peptic ulcer or to produce analgesia which comprises administering an effective amount of a compound of formula II 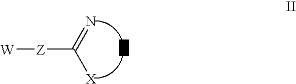
wherein W is optionally substituted aryl; —CHR¹R² where R¹ and R² are independently selected from hydrogen, optionally substituted C₁-C₆ alkyl, optionally substituted C₃-C₇ cycloalkyl and optionally substituted aryl or R¹ and R² are linked to form an optionally substituted C₅-C₇ cycloalkyl; OR′ where R′ is optionally substituted aryl; optionally substituted C₃-C₇ cycloalkyl; or optionally substituted C₁-C₆ alkyl; provided that R¹ and R² are not both hydrogen;
Z is imino, C₁-C₂ alkylene, —CH₂NH— or —CH₂CH₂NH—;
X is O or S; and
Y is optionally substituted C₂-C₃ alkylene; provided that W is not OR′ when Z is imino or —CH₂NH—; and
with the further provisos that
a) when Y is CH₂CH₂, X is O and Z is imino then
(i) W is not unsubstituted or 2-mono-, 2,2-di, 2,5-di, 2,6-di or 2,4,6-tri C₁₋₃ alkyl substituted cyclohexyl or 2-mono- or 2,5,-di C₁₋₃ alkyl substituted cyclopentyl or 2-C₁₋₃ alkyl substituted cycloheptyl; and
(ii) if W is CHR¹R² and R¹ is H then R² is not selected from phenyl; phenyl substituted with methoxy, Br. C1, F or trifluoromethyl; 3-nitrophenyl; 3- or 4-methylphenyl; 2- or 4-bromomethylphenyl; 2- or 4-chloromethylphenyl; or 2,3- or 2,6 dimethylphenyl; and
(iii) if W is CHR¹R² and R¹ is CH₃ or cyclopropyl then R¹ is not phenyl or phenyl substituted with alkyl, halomethyl, fluoro or trifluoromethyl; and
b) when Y is (CH₂)₂₋₄, X is O or S, Z is imino and W is CHR¹R², then
(i) if R¹ is CF₃, CF₂CF₃ or CF₂CF₂CF₃ then R² is not alkyl, optionally substituted cycloalkyl or optionally substituted aryl, and
(ii) if R¹ is optionally substituted cyclopropyl, R² is not H, alkyl or optionally substituted cyclopropyl;
or a pharmaceutically acceptable ester or salt thereof, to a subject in need thereof.

49. The method according to claim 46, wherein the disease is a degenerative condition selected from the group consisting of stroke, aging, ischemia, and CNS trauma.

50. The method according to claim 46, wherein the disease is a neurodegenerative disease selected from the group consisting of Alzheimer's disease and Parkinson's disease.

51. The method according to claim 45, 47 or 48, wherein W is aryl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₁-C₆ alkynyl or aryloxy); C₅-C₆ cycloalkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy); —CHR¹R² where R¹ and R² are independently selected from hydrogen, C₁-C₆ alkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy), C₃-C₆ cycloalkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy) and aryl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy); OR′ where R′ is aryl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy); C₃-C₆ cycloalkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy); or C₁-C₆ alkyl (optionally substituted with hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl or aryloxy).

52. The method according to claim 45, 47 or 48, wherein W is phenyl, cyclohexyl or naphthyl, each of which may be optionally substituted with one to three substituents selected from hydroxy, methoxy, ethoxy, benzyloxy, NO₂, NH₂, halogen, methyl and ethyl; or —CHR¹R² where R¹ and R² are independently selected from phenyl, naphthyl, cyclohexyl, cyclopentyl, cyclobutyl, cyclopropyl, methyl, ethyl, propyl and butyl, each of which may be optionally substituted with hydroxy, methoxy, ethoxy, benzyloxy, NO₂, NH₂, halogen, methyl and ethyl.

53. The method according to claim 45, 47 or 48, wherein Z is imino or —CH₂CH₂NH—.

54. The method according to claim 45, 47 or 48, wherein Y is C₂-C₃ alkylene optionally substituted with C₁-C₄ alkyl, C₃-C₆ cycloalkyl, C₁-C₆ alkanoyloxy or C₁-C₆ alkyloxycarbonyl, or with two substituents which join together to form a 5-6 membered carbocyclic or heterocyclic ring.

55. The method according to claim 54, wherein Y is unsubstituted C₂-C₄ alkylene.

56. The method according to claim 54, wherein Y is ethylene.

57. The method according to claim 45, 47 or 48, wherein the compound of formula II is a compound of formula III: 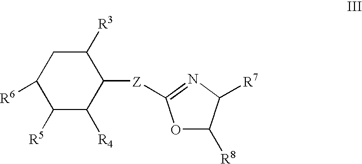
wherein R³, R⁴, R⁵ and R⁶ are independently selected from hydrogen, hydroxy, C₁-C₆ alkyl, C₁-C₆ alkoxy, NO₂, NH₂, C₁-C₆ haloalkyl, halogen, C₃-C₆ cycloalkyl, aryl, C₂-C₆ alkenyl, C₂-C₆ alkynyl and aryloxy;
Z is imino, C₁-C₂ alkylene, or —CH₂CH₂NH—;
R⁷ and R⁸ are independently selected from hydrogen, C₁-C₄ alkyl, C₃-C₆ cycloalkyl, C₁-C₆ alkanoyloxy and C₁-C₆ alkyloxycarbonyl, or R⁷ and R⁸ may together form a 5 or 6 membered aromatic or non-aromatic carbocyclic or heterocyclic ring;
a compound of formula IV: 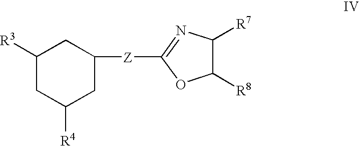
where R³, R⁴, R⁷, R⁸ and Z are as defined in relation to formula III;
a compound of formula V: 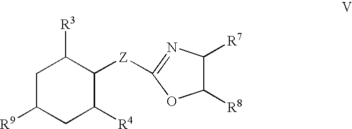
where R³, R⁴, R⁷ and Z are as defined in relation to formula III, and R⁹ is C₁-C₄ alkyl or C₁-C₄ alkoxy;
a compound of formula VI: 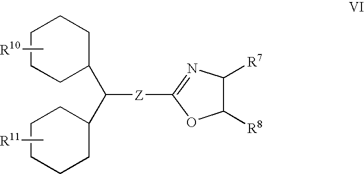
where R⁷, R⁸ and Z are as defined in relation to formula III and R¹⁰ and R¹¹ are independently selected from hydrogen, C₁-C₆ alkyl, C₁-C₆ alkoxy, NO₂, NH₂, C₁-C₆ haloalkyl, halogen, C₃-C₆ cycloalkyl, aryl, C₃-C₆ alkenyl, C₂-C₆ alkynyl and aryloxy;
a compound of formula VII 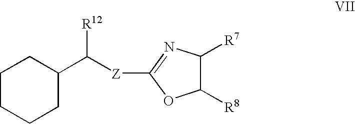
where R⁷, R⁸ and Z are as defined in relation to formula III and R¹² is hydrogen optionally substituted C₁-C₆ alkyl, optionally substituted C₃-C₇ cycloalkyl or optionally substituted aryl; a
compound of formula VIII: 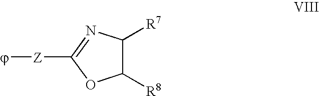
where φ is optionally substituted aryl and R⁷, R⁸ and Z are defined in relation to formula III;
a compound of formula IX: 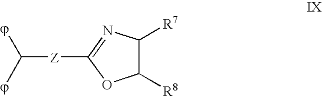
where R⁷, R⁸ and Z and φ are as defined in relation to formula VIII; or
a compound of formula X: 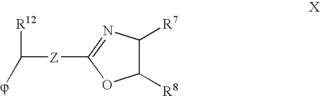
where R⁷, R⁸, R¹² and Z are as defined in relation to formula VII and φ is as defined in relation to formula IX.
